(19) United States
(12) Patent Application Publication
(10) Pub. No.: US 2026/0216183 A1
(43) Pub. Date: Jul. 30, 2026

(54) USE OF COMPOUND 1 IN TREATMENT OR PREVENTION OF ATOPIC DERMATITIS

(71) Applicant: SHENZHEN NEWDEL BIOTECH CO., LTD., Shenzhen, Guangdong (CN)

(72) Inventors: Xun HE, Shenzhen, Guangdong (CN); Feng JIN, Shenzhen, Guangdong (CN); Yuhan GUO, Shenzhen, Guangdong (CN); Tianyuan ZHANG, Shenzhen, Guangdong (CN); Zhenwei LI, Shenzhen, Guangdong (CN); Ying ZHANG, Shenzhen, Guangdong (CN); Wei HU, Shenzhen, Guangdong (CN)

(21) Appl. No.: 19/147,133

(22) PCT Filed: Jan. 12, 2024
(86) PCT No.: PCT/CN2024/071952

§ 371 Date: Jul. 10, 2025

(30) Foreign Application Priority Data
Jan. 13, 2023 (CN) 202310072446.4

(51) Int. Cl. A61K 31/5025; A61P 17/00

(52) U.S. Cl. CPC A61K 31/5025; A61P 17/00

(57) ABSTRACT

The present invention relates to use of N-(3-chloro-5-(trifluoromethyl)phenyl)-3-((6-(4-hydroxypiperidin-1-yl)imidazo[1,2-b]pyridazin-3-yl)ethynyl)-2-methylbenzamide (also referred to herein as compound 1) or a pharmaceutically acceptable salt thereof in the treatment or prevention of atopic dermatitis.

### CROSS-REFERENCE TO RELATED APPLICATIONS

This application is a National Stage Application of PCT application No. PCT/CN2024/071952, filed on Jan. 12, 2024, which claims the priority and benefit of Chinese patent applications No. 202310072446.4, filed on Jan. 13, 2023. The entireties of PCT application No. PCT/CN2024/071952 and Chinese patent applications No. 202310072446.4 are hereby incorporated by reference herein and made a part of this specification.

### TECHNICAL FIELD

The present invention relates to use of N-(3-chloro-5-(trifluoromethyl)phenyl)-3-((6-(4-hydroxypiperidin-1-yl)imidazo[1,2-b]pyridazin-3-yl)ethynyl)-2-methylbenzamide (also referred to herein as compound 1) or a pharmaceutically acceptable salt thereof in the treatment or prevention of atopic dermatitis.

### BACKGROUND ART

Atopic dermatitis (AD) is a common, chronic, recurrent, inflammatory skin disease. The main symptoms are skin dryness, chronic eczematous skin damage, and severe itching. Diseases such as insomnia, anxiety, and depression are even caused in moderate and severe patients, and the life quality is seriously influenced. The disease often combines allergic asthma and allergic rhinitis to occur simultaneously, and is a systemic autoimmune disease.

AD treatment is largely divided into two major categories, topical treatment and systemic treatment. Topical glucocorticoid is a first-line therapy for AD treatment but cannot be used for a long time. Long-term use of glucocorticoids can cause local side effects such as skin atrophy, telangiectasia, pigmentation, and secondary infection, and long-term use of glucocorticoids for a large area, particularly when the skin is damaged, can also cause the inhibition of Hypothalamic-pituitary-adrenal (HPA) axis, which is manifested by pseudo-Cushing's syndrome, steroid diabetes, osteoporosis, exacerbation of infection, induction or exacerbation of ulcer of digestive tract, and induction of mental symptoms. Topical glucocorticoids have strict time limits. Systemic application of glucocorticoids should be avoided as much as possible or minimized. Although dupilumab and Jak inhibitors change the situation where some severe patients have no available medication, nearly 50% of the severe patients do not achieve effective remission and they have non-negligible side effects. For example, dupilumab has a side effect of conjunctivitis, and the Jak inhibitor ruxolitinib carries a black box warning on its product label, alerting to serious side effects including the risk of severe infections; the formation of non-melanoma skin cancers; thrombosis; thrombocytopenia, anemia, and neutropenia occur. There is still a significant unmet clinical need in the field of AD disease and there is an urgent need to develop safer and more effective drugs that can be used for a long period of time.

At present, two marketed drugs of the TRK inhibitor (larotrectinib and entrectinib) are both targeted at tumor patients with NTRK gene mutations. There are no marketed drugs for atopic dermatitis indications. Three drugs are currently in clinical trial phase of atopic dermatitis, which are the TRKA inhibitor Pegcantratinib originally researched by Cephalon company in clinical phase II (terminated), the pan-TRK inhibitor BEN-2293 (capable of inhibiting TRKA, TRKB, and, TRKC) by BenevolentAI company in clinical phase II, and the pan-TRK inhibitor PBI-100 by pyrimad company in clinical phase I, respectively. The pegcantratinib atopic dermatitis clinical study had been terminated in 2010, and BEN-2293 and PBI-100 were still in progress. It can be seen that inhibitors specific for TRKA are at risk of failure in treating atopic dermatitis. No drugs against atopic dermatitis were entered into clinical trials for the DDR1 and DDR2 inhibitors, and preclinical studies of DDRs for this indication were also blank. Also, there are no drugs in preclinical and clinical stages against atopic dermatitis for CSF1R inhibitors.

Short-term stimulation with hapten OXA (oxazolone) can cause contact allergic dermatitis that is mainly mouse Th1 cellular immunity, but continuous and repeated contact with the hapten can induce the mice to generate Th2 skin inflammation similar to human atopic dermatitis. The local skin can be seen with obvious erythema, scale and skin thickening. Histopathology shows epidermal thickening, edema, and inflammatory cell infiltration of a large amount of CD4+ T lymphocytes, mast cells, and the like in the dermis. It is one of the most common models for researching the pathological mechanism of atopic dermatitis and screening for therapeutic drugs. The present invention uses this model to identify the therapeutic effect of the compound on atopic dermatitis.

CN113831344B discloses N-(3-chloro-5-(trifluoromethyl)phenyl)-3-((6-(4-hydroxypiperidin-1-yl)imidazo[1,2-b]pyridazin-3-yl)ethynyl)-2-methylbenzamide.

### SUMMARY

The present invention relates to use of N-(3-chloro-5-(trifluoromethyl)phenyl)-3-((6-(4-hydroxypiperidin-1-yl)imidazo[1,2-b]pyridazin-3-yl)ethynyl)-2-methylbenzamide (also referred to herein as compound 1) or a pharmaceutically acceptable salt thereof in the treatment or prevention of atopic dermatitis. In the present invention, atopic dermatitis includes infant atopic dermatitis, childhood atopic dermatitis, and adolescent/adult atopic dermatitis classified by age characteristics; mild atopic dermatitis, moderate atopic dermatitis, and severe atopic dermatitis by severity; Th2 type, Th2/Th17 mixed type, and Th2/Th22 mixed type atopic dermatitis based on the inflammation type.

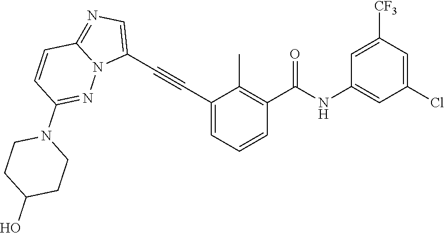 [SMILES: Cc1c(C#Cc2cnc3ccc(N4CCC(O)CC4)nn23)cccc1C(=O)Nc1cc(Cl)cc(C(F)(F)F)c1]

In a first aspect, the present invention provides use of compound 1 or a pharmaceutically acceptable salt thereof in the manufacture of a medicament for treating or preventing atopic dermatitis.

In one embodiment of the first aspect, the medicament is administered topically. In another embodiment of the first aspect, the medicament is administered orally.

In a second aspect, the present invention provides a method for treating or preventing atopic dermatitis, comprising administering to a subject in need thereof a therapeutically effective amount of compound 1 or the pharmaceutically acceptable salt thereof.

In one embodiment of the second aspect, compound 1 or the pharmaceutically acceptable salt thereof is administered topically. In another embodiment of the second aspect, compound 1 or the pharmaceutically acceptable salt thereof is administered orally.

In a third aspect, the present invention provides a pharmaceutical composition for treating or preventing atopic dermatitis, comprising compound 1 or the pharmaceutically acceptable salt thereof, and a pharmaceutically acceptable excipient.

In one embodiment of the third aspect, the pharmaceutical composition is administered topically. In another embodiment of the third aspect, the pharmaceutical composition is administered orally.

In a fourth aspect, the present invention provides compound 1 or a pharmaceutically acceptable salt thereof for use in the treatment or prevention of atopic dermatitis.

In one embodiment of the fourth aspect, compound 1 or the pharmaceutically acceptable salt thereof is administered topically. In another embodiment of the fourth aspect, compound 1 or the pharmaceutically acceptable salt thereof is administered orally.

### BRIEF DESCRIPTION OF THE DRAWINGS

FIG. 1 shows the trend of changes in the body weight of mice from day 0 to day 26 of the smearing administration experiment.

FIG. 2 shows the thickness of the pinna of the mice from day 0 to day 26 of the smearing administration experiment.

FIG. 3A shows HE staining pictures of mouse pinna tissue sections on day 26 of the smearing administration experiment at 20× magnification. FIG. 3B shows statistical analysis of the thickness of the mouse pinna epidermal tissue on day 26.

FIG. 4 shows inflammatory cell infiltration in the mouse pinna tissue on day 26 of the smearing administration experiment. FIG. 4A is a histopathological image and FIG. 4B is scores of inflammatory cell infiltration.

FIG. 5 shows the thickness of dorsal skin of the mice on day 26 of the smearing administration experiment.

FIG. 6 shows clinical scores of dorsal skin of the mice from day 0 to day 26 of the smearing administration experiment.

FIG. 7 shows a line graph showing the rate of changes in the body weight of mice from day 0 to day 21 of the oral administration experiment.

FIG. 8 shows a line graph showing the rate of changes in the thickness of the pinna of mice from day 0 to day 21 of the oral administration experiment.

FIG. 9 shows a line graph showing clinical scores of back of mice from day 0 to day 21 of the oral administration experiment.

FIG. 10 shows a representative graph showing H&E staining of right ear tissue of mice of the oral administration experiment.

FIG. 11 shows a statistical graph showing the thickness of the epidermal layer of the right ear of mice of the oral administration experiment.

FIG. 12 shows a statistical graph showing inflammatory cell infiltration in the right ear tissue of mice of the oral administration experiment.

FIG. 13 shows a representative graph showing Masson staining of the dermis layer of mice of the oral administration experiment.

FIG. 14 shows a statistical graph showing the thickness of the dermis layer of the right ear of mice of the oral administration experiment.

### DETAILED DESCRIPTION

The present invention is further illustrated by, but not limited to, the following examples that explain the present invention.

### Example 1: Inhibitory Effect of Compound 1 on Kinase Activities of TRK and DDR

### Assay Principle of HTRF Method:

A kinase phosphorylates a substrate, and an antibody labeled with Eu-CryPtate binds to a phosphorylation site in the substrate; streptavidin-XL665 binds to the substrate biotin; when Eu and XL665 are close, Eu as a donor emits emission light (620 nm) after being excited by a light source (320 nm) and transfers energy resonance to a close XL665 receptor, and the receptor emits emission light (665 nm) after being excited; the specific signal is proportional to the phosphorylated substrate; when an inhibitor is added, the phosphorylation level is inhibited, the emission light at 665 nm is undetectable, and only the emission light at 620 nm is detected, so as to evaluate the level of inhibition of kinase activity by the compound.

### TRKA Enzyme Activity Assay:

Compound 1 was serially diluted in DMSO from a starting concentration of 1 μM to obtain a total of 10 concentrations after four-fold dilution. Replicate wells were set for the assay. 25 nL of the compound was transferred to a 384 reaction plate (Catalog No. 784075, Greiner) using Echo 665. 2× kinase solution was prepared with 1× kinase reaction buffer (5× buffer, 5 mM MgCl₂, 1 mM DTT, H₂O, and 1 mM MnCl₂). 2.5 μL of TRKA (1.5 nM, 08-186, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) (Catalog No. 61TK0BLE, Cisbio-PerkinElmer) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### TRKB Enzyme Activity Assay:

Compound 1 was serially diluted in DMSO from a starting concentration of 1 μM to obtain a total of 10 concentrations after four-fold dilution. Replicate wells were set for the assay. 25 nL of the compound was transferred to a 384 reaction plate using Echo 665. 2× kinase solution was prepared with 1× kinase reaction buffer (5× buffer, 5 mM MgCl₂, 1 mM DTT, H₂O, and 1 mM MnCl₂). 2.5 μL of TRKB (1.4 nM, 08-187, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### TRKC Enzyme Activity Assay:

Compound 1 was serially diluted in DMSO from a starting concentration of 1 μM to obtain a total of 10 concentrations after four-fold dilution. Replicate wells were set for the assay. 2× kinase solution was prepared with 1× kinase reaction buffer (5× buffer, 5 mM MgCl₂, 1 mM DT, H₂O, and 1 mM MnCl₂). 2.5 μL of TRKC (1.5 nM, 08-197, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### DDR1 Enzyme Activity Assay:

Compound 1 was serially diluted in DMSO from a starting concentration of 1 μM to obtain a total of 10 concentrations after four-fold dilution. Replicate wells were set for the assay. 2× kinase solution was prepared with 1× kinase reaction buffer (5× buffer, 5 mM MgCl₂, 1 mM DTT, H₂O, and 1 mM MnCl₂). 2.5 μL of DDR1 (2.7 nM, 08-113, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### DDR2 Enzyme Activity Assay:

Compound 1 was serially diluted in DMSO from a starting concentration of 1 μM to obtain a total of 10 concentrations after four-fold dilution. Replicate wells were set for the assay. 2× kinase solution was prepared with 1× kinase reaction buffer (5× buffer, 5 mM MgCl₂, 1 mM DT, H₂O, and 1 mM MnCl₂). 2.5 μL of DDR2 (1.3 nM, 08-114, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

### CSF1R Enzyme Activity Assay:

Compound 1 was serially diluted in DMSO from a starting concentration of 10 μM to obtain a total of 10 concentrations after four-fold dilution. Replicate wells were set for the assay. 2× kinase solution was prepared with 1× kinase reaction buffer (5× buffer, 5 mM MgCl2, 1 mM DTT, H2O, and 1 mM MnCl2). 2.5 μL of CSF1R (1.3 nM, 08-114, Carna) solution was transferred to a 384 reaction plate. The plate was centrifuged at 1000 rpm for 60 s using a centrifuge and incubated at 25° C. for 10 min. A mixed solution of 2× biotin-labeled tyrosine kinase substrate (1 μM) and ATP (25 μM) was prepared using the kinase reaction buffer. 2.5 μL of the mixed solution of the substrate and ATP was added to the reaction plate to start the reaction, and the plate was centrifuged at 1000 rpm for 60 s using the centrifuge. The plate was sealed with a plate-sealing film and incubated at 25° C. for 40 min. 5 μL of a mixed solution of Eu-CryPtate-labeled anti-tyrosine kinase antibody and streptavidin-XL665 was added to each well of the reaction plate. The plate was centrifuged at 1000 rpm for 1 min and incubated at 25° C. for 60 min. The fluorescence signals at 620 nm (Cryptate) and 665 nm (XL665) were read using a BMG microplate reader. The ratio of the fluorescence signals, i.e., (665 nm/620 nm)×10000, in each well was calculated, and the ratio represented the degree of the kinase activity.

The IC₅₀ of compound 1 to TRKA, TRKB, TRKC, DDR1, DDR2, and CSF1R obtained from the assays described above are listed in Table 1.

TABLE 1
Inhibitory effect of compound 1 on
kinase activities of TRK and DDR

| | IC₅₀ (nM) |
|---|---|
| TRKA | 4.397 |
| TRKB | 0.4325 |
| TRKC | 0.350 |
| DDR1 | 0.7616 |
| DDR2 | 37.93 |
| CSF1R | 14.71 |

### Example 2: Preparation of Compound 1 Ointment

The components of the formula are 8 mg/g of a free base compound 1 according to the total weight of the formula, DMSO accounting for 5% of the excipient, PEG4000 accounting for 20% of the excipient, and PEG400 accounting for 75% of the excipient. PEG4000 and part of PEG400 were placed in a container, mixed, and heated in a water bath (oil bath) at 80° C. to melt. The mixture was stirred continuously during heating, and cooled to 60° C. or less after being mixed well to give a mixed solution I; compound 1 powder and DMSO were weighed and placed in the same container, compound 1 was dissolved by ultrasonication, the remaining PEG400 was added, and the mixture was mixed well to give a mixed solution H; the mixed solution II was added to the mixed solution I, and the mixture was mixed well, placed in a container, cooled, and sealed to give an ointment (hereinafter also referred to as “test drug”).

### Example 3: Evaluation of Therapeutic Effect of Compound 1 Ointment on OXA-Induced Atopic Dermatitis Mouse Model

### Method

C57BL/6 mice (female mice, weighed about 18.0 g, 6-8 weeks old) were obtained from Shanghai Model Organisms Center, Inc. and divided equally according to body weight, which were groups receiving the following treatments:
Group 1: 1% oxazolone (abbreviated as OXA)+vehicle (abbreviated as group 1);
Group 2: 1% OXA+dexamethasone ointment (abbreviated as DEX) (abbreviated as group 2);
Group 3: 1% OXA+test drug (abbreviated as group 3).

On day 0, the backs of the mice of each group were depilated with depilatory cream. The backs and the right ears of the mice of each group were smeared with 1% OXA. The 1% OXA solution was prepared by weighing 0.1 g of OXA powder, adding 10 mL of an acetone solution, mixing well, and dissolving. After induction, the mice of each group were normally raised for 7 days. The backs and the right ears of the mice of each group were smeared with 1% OXA at 40 mg/kg on days 7, 10, 12, 14, 17, 19, 21, and 24. The mice in group 1 were smeared with the vehicle every day after induction with 1% OXA; mice in group 2 were dosed with DEX cream (compound dexamethasone acetate cream, 0.75 mg/g, NMPA Drug Approval No. H44034170, China Resources Sanjiu Medical & Pharmaceutical Co., Ltd.) twice daily after induction with 1% OXA by smearing the backs and right ears of the mice with 11.25 mg/kg of the cream; mice in group 3 were treated with 100 mg/kg of the test drug (8 mg/g compound 1) by smearing twice daily after induction with 1% OXA. Mice were weighed three times a week, the thicknesses of the right ears of the mice were measured, and clinical scores were made on the back in terms of three aspects of erythema (0-3 points), skin thickness (0-3 points), and scab (0-3 points), with the clinical score being the sum of the three items. The mice were sacrificed on day 26. The mouse right ear samples were fixed with 4% PFA and subjected to paraffin embedding, tissue section, and HE staining. The thickness of the epidermal layer was measured under a microscope, and inflammatory cell infiltration was scored.

### Result

The weight fluctuation of the mice in each group is shown in Table 2 and FIG. 1. The weight increase of group 2 positive drug DEX group is significantly inferior to that of group 1 vehicle group (p<0.05) from day 14 to day 17. The weight increase of group 3 test drug group is significantly superior to that of group 1 vehicle group from day 14 to day 21. The weight increase of group 3 test drug group is significantly superior to that of the positive drug group DEX group from day 14 to day 21. The statistical details are shown in Table 3.

TABLE 2
Mouse average body weight (g)

Group

| Time (days) | Group 1 | Group 2 | Group 3 |
|---|---|---|---|
| 0 | 17.94 ± 0.37 | 17.36 ± 0.38 | 17.82 ± 0.25 |
| 4 | 18.74 ± 0.42 | 17.96 ± 0.65 | 18.40 ± 0.54 |
| 7 | 19.76 ± 0.34 | 19.18 ± 0.47 | 19.66 ± 0.27 |
| 10 | 17.20 ± 0.56 | 17.32 ± 0.37 | 18.22 ± 0.18 |
| 12 | 18.20 ± 0.64 | 17.80 ± 0.44 | 19.06 ± 0.27 |
| 14 | 19.43 ± 0.54 | 16.96 ± 0.49 | 20.10 ± 0.31 |
| 17 | 19.60 ± 0.51 | 16.86 ± 0.30 | 20.02 ± 0.30 |
| 19 | 19.30 ± 0.75 | 18.06 ± 0.45 | 20.76 ± 0.38 |
| 21 | 18.80 ± 0.65 | 18.08 ± 0.65 | 20.96 ± 0.43 |
| 24 | 20.43 ± 0.84 | 18.64 ± 0.54 | 20.36 ± 0.34 |
| 26 | 20.90 ± 0.96 | 19.56 ± 0.46 | 21.16 ± 0.52 |

TABLE 3
Two way Anova analysis of statistical data of
mouse body weight over time between groups

Group

| | Group 1 vs. | Group 1 vs. | Group 2 vs. |
|---|---|---|---|
| Time (days) | group 2 | group 3 | group 3 |
| 0 | ns | ns | ns |
| 4 | ns | ns | ns |
| 7 | ns | ns | ns |
| 10 | ns | ns | ns |
| 12 | ns | ns | ns |
| 14 | * | ** | ** |
| 17 | * | *** | *** |
| 19 | ns | ** | ** |
| 21 | ns | * | * |
| 24 | ns | ns | ns |
| 26 | ns | ns | ns |

ns: no significant difference
* p < 0.05
** p < 0.01
*** p < 0.001

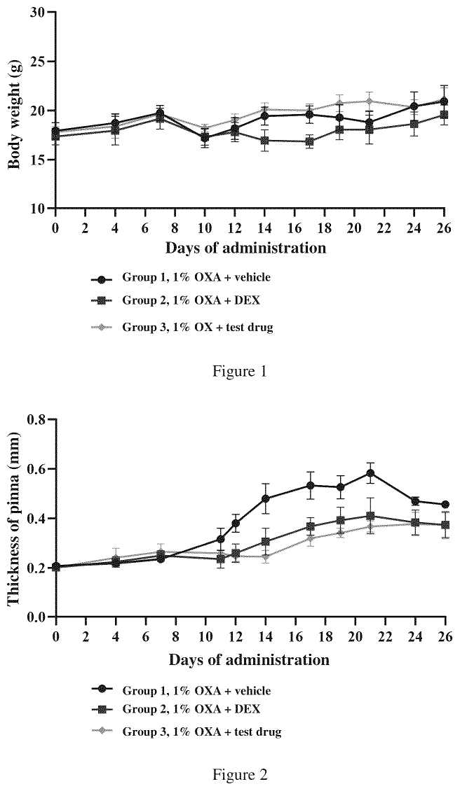

The increase in the thickness of the pinna of the right ear of the mice from day 0 to day 26 is shown in Table 4 and FIG. 2. Statistical analysis was performed on each group using two way ANOVA. As in Table 5, statistical differences occurred between group 2 DEX group and group 1 vehicle group from day 12 to day 26; statistical differences occurred between group 3 test drug group and group 1 vehicle group from day 12 to day 26. Group 3 test drug group was not statistically different from group 2 DEX group, and they had similar inhibitory effects on the increase in the thickness of the pinna.

TABLE 4
Thickness of pinna of mice (mm)

Group

| Time (days) | Group 1 | Group 2 | Group 3 |
|---|---|---|---|
| 0 | 0.21 ± 0.00 | 0.20 ± 0.00 | 0.20 ± 0.00 |
| 4 | 0.22 ± 0.00 | 0.22 ± 0.01 | 0.24 ± 0.02 |
| 7 | 0.24 ± 0.01 | 0.25 ± 0.01 | 0.27 ± 0.01 |
| 10 | 0.32 ± 0.03 | 0.24 ± 0.02 | 0.26 ± 0.01 |
| 12 | 0.38 ± 0.02 | 0.26 ± 0.02 | 0.25 ± 0.01 |
| 14 | 0.48 ± 0.04 | 0.31 ± 0.02 | 0.25 ± 0.01 |
| 17 | 0.53 ± 0.03 | 0.37 ± 0.02 | 0.32 ± 0.01 |
| 19 | 0.53 ± 0.03 | 0.39 ± 0.02 | 0.34 ± 0.01 |
| 21 | 0.58 ± 0.02 | 0.41 ± 0.03 | 0.37 ± 0.01 |
| 24 | 0.47 ± 0.01 | 0.38 ± 0.02 | 0.38 ± 0.02 |
| 26 | 0.46 ± 0.01 | 0.37 ± 0.02 | 0.37 ± 0.02 |

TABLE 5
Two way Anova analysis of statistical data or thickness
of pinna of mice over time between groups

Group

| | Group 1 vs. | Group 1 vs. | Group 2 vs. |
|---|---|---|---|
| Time (days) | group 2 | group 3 | group 3 |
| 0 | ns | ns | ns |
| 4 | ns | ns | ns |
| 7 | ns | ns | ns |
| 10 | ns | ns | ns |
| 12 | * | * | ns |
| 14 | * | * | ns |
| 17 | * | * | ns |
| 19 | * | * | ns |
| 21 | * | * | ns |
| 24 | * | * | ns |
| 26 | | | |

ns: no significant difference
* p < 0.05

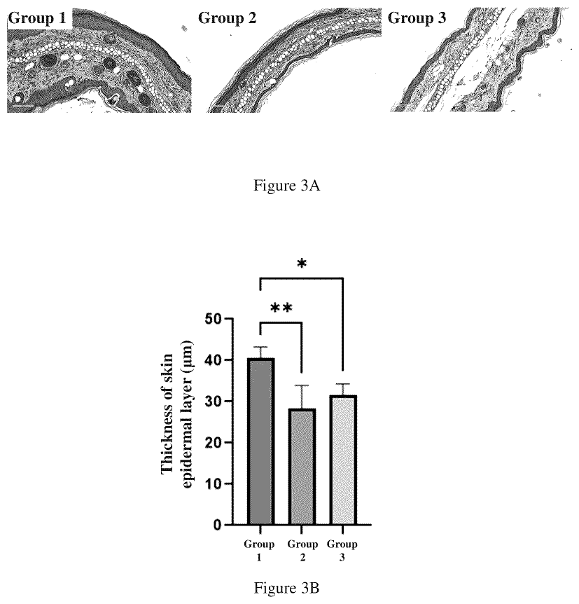

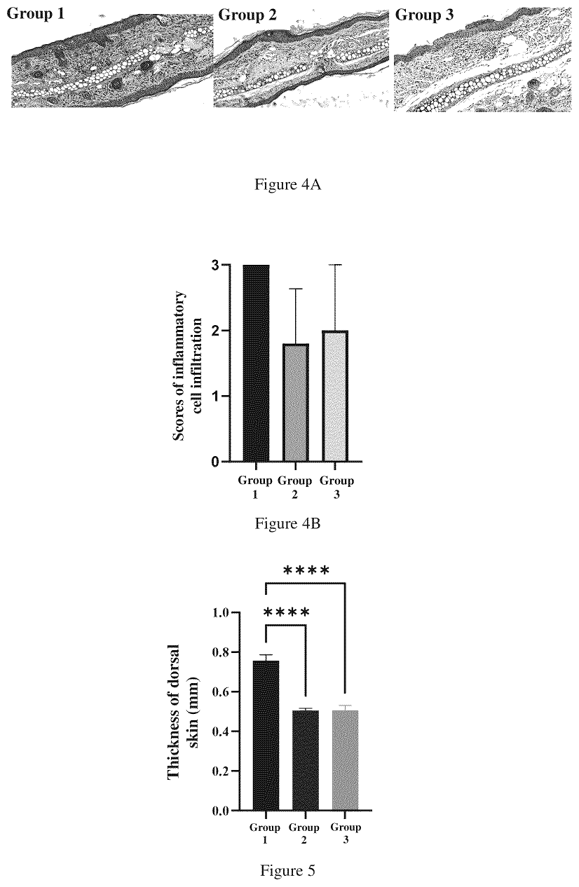

On day 26, pathological analysis was carried out on the section of the pinna of the mice (FIG. 3A), and the numerical value of the thickness of the epidermal layer was as follows: group 1 vehicle group was 40.54 μm±2.73 μm, group 2 DEX group was 28.34±5.66 μm, and group 3 test drug group was 31.56±2.78 μm, as shown in FIG. 3B. According to one way ANOVA statistical analysis, the thickness of the epidermal layer of group 2 positive drug group was significantly reduced (p<0.01) relative to group 1 vehicle group, and the thickness of the epidermal layer of group 3 test drug group was also significantly reduced (p<0.05). The efficacy was significant. Inflammatory cell infiltration is shown in FIG. 4A, and the score is shown in FIG. 4B (infiltration proportion of inflammatory cells in the dermis layer<5% is rated as 0 point, <33% is 1 point mild, <67% is 2 points moderate, and >67% is 3 points severe). The positive drug group and the test drug group inhibited the infiltration degree of inflammatory cells relative to the vehicle group.

When the experiment was performed to day 26, mice in each group were euthanized, and the dorsal skin was taken for thickness measurement. The numerical values of each group of groups 1-3 are 0.76±0.02 mm, 0.51±0.01 mm, and 0.51±0.01 mm, respectively (FIG. 5). According to one way ANOVA statistical analysis, statistical differences occurred in group 2 and group 3 (P<0.0001) compared to group 1 vehicle group. Group 2 and group 3 were with no statistical difference, and both had similar efficacy against dorsal skin thickness inhibition.

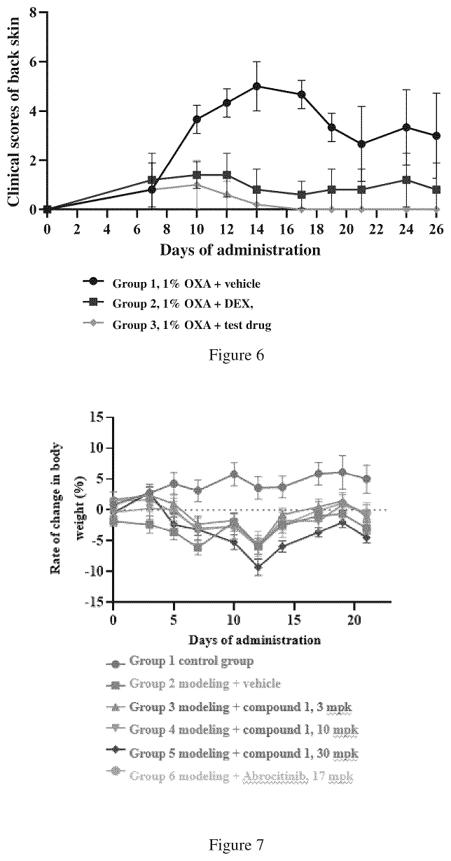

Clinical scores of dorsal skin of the mice from day 0 to day 26 are shown in Table 6 and FIG. 6. According to statistical analysis of two way ANOVA, as in Table 7, statistical differences occurred between group 2 dexamethasone group and vehicle group from day 7 to day 19; statistical differences occurred between group 3 test drug group and vehicle group from day 7 to day 19. Group 2 dexamethasone group and group 3 test drug group had no statistical difference, and they had similar drug effect on inhibiting clinical symptoms of dorsal skin of the mice.

TABLE 6
Clinical scores of mouse back

Group

| Time (days) | Group 1 | Group 2 | Group 3 |
|---|---|---|---|
| 0 | 0.00 ± 0.00 | 0.00 ± 0.00 | 0.00 ± 0.00 |
| 7 | 0.80 ± 0.49 | 1.20 ± 0.49 | 0.80 ± 0.49 |
| 10 | 3.67 ± 0.33 | 1.40 ± 0.24 | 1.00 ± 0.45 |
| 12 | 4.33 ± 0.33 | 1.40 ± 0.40 | 0.60 ± 0.24 |
| 14 | 5.00 ± 0.58 | 0.80 ± 0.37 | 0.20 ± 0.20 |
| 17 | 4.67 ± 0.33 | 0.60 ± 0.24 | 0.00 ± 0.00 |
| 19 | 3.33 ± 0.33 | 0.80 ± 0.37 | 0.00 ± 0.00 |
| 21 | 2.67 ± 0.88 | 0.80 ± 0.37 | 0.00 ± 0.00 |
| 24 | 3.33 ± 0.88 | 1.20 ± 0.49 | 0.00 ± 0.00 |
| 26 | 3.00 ± 1.00 | 0.80 ± 0.49 | 0.00 ± 0.00 |

TABLE 7
Two way Anova analysis of statistical data of clinical scores
of dorsal skin of the mice over time between groups

Group

| | Group 1 vs. | Group 1 vs. | Group 2 vs. |
|---|---|---|---|
| Time (days) | group 2 | group 3 | group 3 |
| 0 | ns | ns | ns |
| 7 | * | ** | ns |
| 10 | ** | ** | ns |
| 12 | * | * | ns |
| 14 | ** | ** | ns |
| 17 | ** | * | ns |
| 19 | ** | ** | ns |
| 21 | ns | ns | ns |
| 24 | ns | ns | ns |
| 26 | ns | ns | ns |

ns: no significant difference
* p < 0.05
** p < 0.01

### CONCLUSION

From pinna thickness measurement, pinna epidermal tissue thickness, pinna tissue inflammatory cell infiltration, dorsal skin thickness, and dorsal skin clinical scores, compound 1 has excellent drug effect on mouse atopic dermatitis model symptoms induced by 1% OXA. Compound 1 has similar drug effect with the positive drug dexamethasone cream, the mouse weight increase is better than that of the dexamethasone cream group, and the safety is better. Long-term use of glucocorticoids can cause side effects such as skin atrophy, telangiectasia, pigmentation, and secondary infection. Glucocorticoids cannot be clinically used for a long time and have strict time use restrictions. Compound 1 provides a safer and more effective new option for atopic dermatitis treatment.

### Example 4: Method for Evaluating Therapeutic Effect of Compound 1 Administered Orally on OXA-Induced Atopic Dermatitis Mouse Model

(1) Screening and grouping: 60 female 6- to 8-week-old Balb/c mice in total were used as experimental animals in this project, which were obtained from Shanghai Model Organisms Center, Inc. The first grouping was done before the start of the experiment. The mice were randomly assigned to a control group (group 1, 10 mice) based on body weight, with the remainder being model groups. On day 8 after sensitization (designated as day 0), the mice in the model group were grouped for the second time, which were randomly divided into 5 groups (group 2, group 3, group 4, group 5, and group 6) of 10 mice each, based on the body weight and the thickness of the pinna of the right ear of the mice.

(2) Animal modeling: The day when the first grouping was done was designated as day (−7). The backs of the mice were depilated with depilatory cream (area of about 2 cm×3 cm). The backs and the right ears of the mice in the model group were sensitized with 40 mg/kg of a solution of 1% OXA in acetone, and the backs and the right ears of the mice in group 1 were smeared with acetone. The 1% OXA solution was prepared by weighing 0.1 g of OXA powder, adding 10 mL of an acetone solution, mixing well, and dissolving. The backs and the right ears of the mice in group 1 were smeared with acetone on days 0, 3, 5, 7, 10, 12, 14, 17, 19, and 21; the backs and the right ears of the mice in group 2, group 3, group 4, group 5, and group 6 were smeared with a solution of 1% OXA in acetone.

(3) The administration mode of the test substance: For 22 consecutive days from day 0 to day 21, mice in group 2 were given vehicle, mice in group 3 to group 5 were intragastrically administered with compound 1 at doses of 3 mpk, 10 mpk, and 30 mpk (based on free base), respectively, once daily for treatment, and mice in group 6 were intragastrically administered with the positive drug Abrocitinib at a dose of 17 mpk once daily for treatment.

(4) Detection indexes: For 22 consecutive days from day 0 today 21, mice were weighed three times a week, the thickness of the pinna of the right ear of the mice was measured three times a week before administration, and scores were made on the back in terms of three aspects of erythema (0-3 points), skin thickness (0-3 points), and scab (0-3 points). The mice were sacrificed on day 21. The skin of the right ear of the mice was left for pathological examination. The thickness of the epidermal layer and inflammatory cell infiltration were measured by H&E staining, and the thickness of the dermis layer was measured by Masson staining.

### Result

The rate of changes in body weight of the mice during administration is shown in FIG. 7. The group 2 model group had significantly reduced body weight relative to the group 1 control group using Two-way ANOVA analysis (****p<0.0001; Table 8). Compared with the model group 2 administered with vehicle, compound 1 could significantly inhibit body weight loss (**p<0.01; Table 8) when it was administered at a dose of 3 mpk by intragastric administration once daily for 22 consecutive days; compound 1 had no significant effect on body weight change (p>0.05; Table 8) when it was administered at a dose of 10 mpk by intragastric administration once daily for 22 consecutive days; compound 1 had no effect on body weight change (p>0.05; Table 8) when it was administered at a dose of 30 mpk by intragastric administration once daily for 22 consecutive days. Compared with the model group 2 administered with vehicle, the positive control drug Abrocitinib had no significant effect on body weight change (p>0.05; Table 8) when it was administered at a dose of 17 mpk by intragastric administration once daily for 22 consecutive days.

TABLE 8
Analysis of rate of change in body weight of mouse

| | | Significance |
|---|---|---|
| Group | p value | level |
| Group 1 control group | <0.0001 | **** |
| Group 2 modeling + vehicle, p.o. | — | — |
| Group 3 modeling + compound 1, 3 mpk, p.o. | 0.0013 | ** |
| Group 4 modeling + compound 1, 10 mpk, p.o. | 0.3653 | ns |
| Group 5 modeling + compound 1, 30 mpk, p.o. | 0.9561 | ns |
| Group 6 modeling + Abrocitinib, 17 mpk, p.o. | 0.0939 | ns |

Note:
The rate of change in body weight in each experimental group was compared using Two-way ANOVA analysis, and the p-values were all compared to that of group 2.

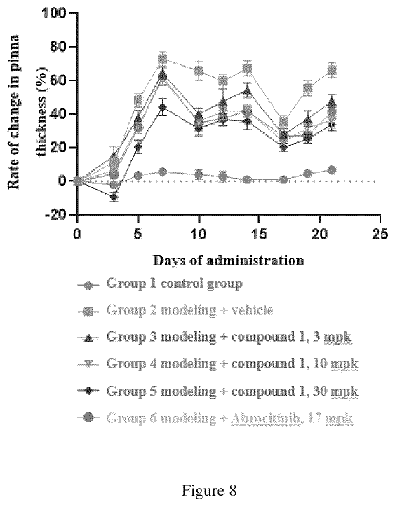

The rate of changes in the thickness of the pinna of the right ear of the mice during administration is shown in FIG. 8. The model group 2 had significantly increased thickness of the pinna of the right ear relative to the group 1 control group using Two-way ANOVA analysis (****p<0.0001; Table 9). Compared with the model group 2 administered with vehicle, compound 1 could significantly reduce the thickness of the pinna of the right ear (*p<0.05; Table 9) when it was administered at a dose of 3 mpk by intragastric administration once daily for 22 consecutive days; compound 1 could significantly inhibit right ear pinna thickening (****p<0.0001; Table 9) when it was administered at a dose of 10 mpk by intragastric administration once daily for 22 consecutive days; compound 1 could significantly reduce the thickness of the pinna of the right ear (****p<0.0001; Table 9) when it was administered at a dose of 30 mpk by intragastric administration once daily for 22 consecutive days, and continued the significant inhibition from day 4 of administration (*p<0.05; Table 10). Compared with the model group 2 administered with vehicle, the positive control drug Abrocitinib could significantly inhibit right ear pinna thickening (****p<0.0001; Table 9) when it was administered at a dose of 17 mpk by intragastric administration once daily for 22 consecutive days.

TABLE 9
Analysis of rate of change in the thickness of the pinna
of the right ear of mice in each experimental group

| | | Significance |
|---|---|---|
| Group | p value | level |
| Group 1 control group | <0.0001 | **** |
| Group 2 modeling + vehicle, p.o. | — | — |
| Group 3 modeling + compound 1, 3 mpk, p.o. | 0.0238 | * |
| Group 4 modeling + compound 1, 10 mpk, p.o. | <0.0001 | **** |
| Group 5 modeling + compound 1, 30 mpk, p.o. | <0.0001 | **** |
| Group 6 modeling + Abrocitinib, 17 mpk, p.o. | <0.0001 | **** |

Note:
The rate of change in the thickness of the pinna of the right ear in each experimental group was compared using Two-way ANOVA analysis, and the p-values were all compared to that of group 2.

TABLE 10
Analysis of rate of change in the thickness of the pinna of the right ear of mice at each time point

| | Group 3 | | Group 4 | | Group 5 | | Group 6 | |
|---|---|---|---|---|---|---|---|---|
| | p | Significance | p | Significance | p | Significance | p | Significance |
| Days | value | level | value | level | value | level | value | level |
| 0 | — | — | — | — | — | — | — | — |
| 3 | 0.9916 | ns | 0.8764 | ns | 0.004 | ** | 0.6553 | ns |
| 5 | 0.548 | ns | 0.06 | ns | 0.0017 | ** | 0.076 | ns |
| 7 | 0.6796 | ns | 0.2777 | ns | 0.0044 | ** | 0.6624 | ns |
| 10 | 0.0156 | * | 0.004 | ** | 0.0015 | ** | 0.0056 | ** |
| 12 | 0.7119 | ns | 0.1282 | ns | 0.0065 | ** | 0.0317 | * |
| 14 | 0.3452 | ns | 0.0069 | ** | 0.0024 | ** | 0.0036 | ** |
| 17 | 0.6602 | ns | 0.1253 | ns | 0.0373 | * | 0.4614 | ns |
| 19 | 0.1103 | ns | 0.0052 | ** | 0.0005 | *** | 0.001 | ** |
| 21 | 0.054 | ns | 0.003 | ** | 0.0005 | *** | 0.0126 | * |

Note:
The rate of change in the thickness of the pinna of the right ear at each time point was compared using Two-way ANOVA analysis, and the p-values were all compared to that of group 2.

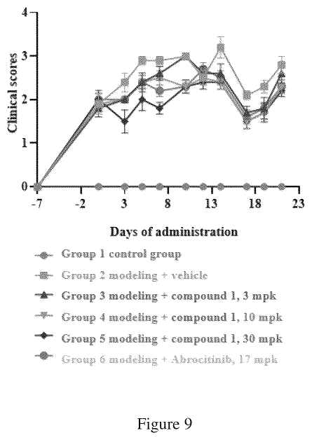

The clinical scores of dorsal skin of the mice during administration is shown in FIG. 9. The model group 2 had significantly increased clinical scores of dorsal skin relative to the group 1 control group using Two-way ANOVA analysis (****p<0.0001; Table 11). Compared with the model group 2 administered with vehicle, compound 1 had no significant inhibition on increased clinical scores of dorsal skin (p>0.05; Table 11) when it was administered at a dose of 3 mpk by intragastric administration once daily for 22 consecutive days; compound 1 could significantly reduce clinical scores of dorsal skin (*p<0.05; Table 11) when it was administered at a dose of 10 mpk by intragastric administration once daily for 22 consecutive days; compound 1 significantly inhibited increased clinical scores of dorsal skin (***p<0.001; Table 11) when it was administered at a dose of 30 mpk by intragastric administration once daily for 22 consecutive days. Compared with the model group G2 administered with vehicle, the positive control drug Abrocitinib could significantly reduce clinical scores of dorsal skin (*p<0.05; Table 11) when it was administered at a dose of 17 mpk by intragastric administration once daily for 22 consecutive days.

TABLE 11
Analysis of clinical scores of dorsal
skin of mice in each experimental group

| | | Significance |
|---|---|---|
| Group | p value | level |
| Group 1 control group | <0.0001 | **** |
| Group 2 modeling + vehicle, p.o. | — | — |
| Group 3 modeling + compound 1, 3 mpk, p.o. | 0.3208 | ns |
| Group 4 modeling + compound 1, 10 mpk, p.o. | 0.0127 | * |
| Group 5 modeling + compound 1, 30 mpk, p.o. | 0.0001 | *** |
| Group 6 modeling + Abrocitinib, 17 mpk, p.o. | 0.0223 | * |

Note:
The clinical scores of dorsal skin in each experimental group were compared using Two-way ANOVA analysis, and the p-values were all compared to that of group 2.

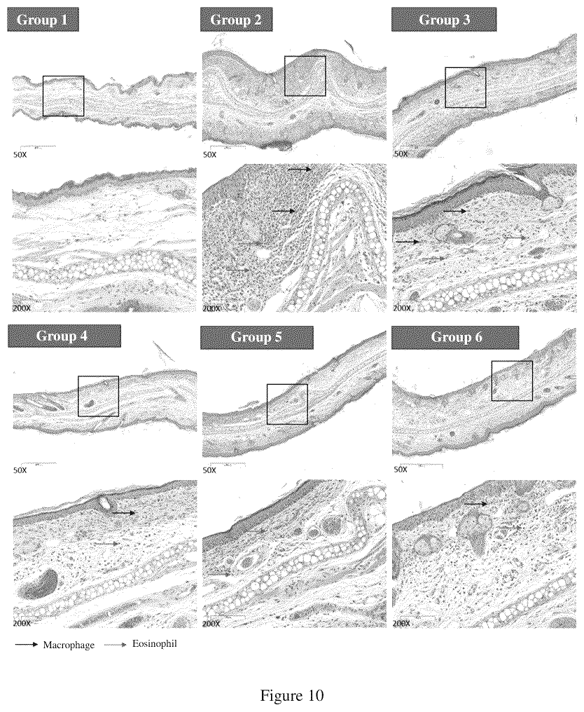

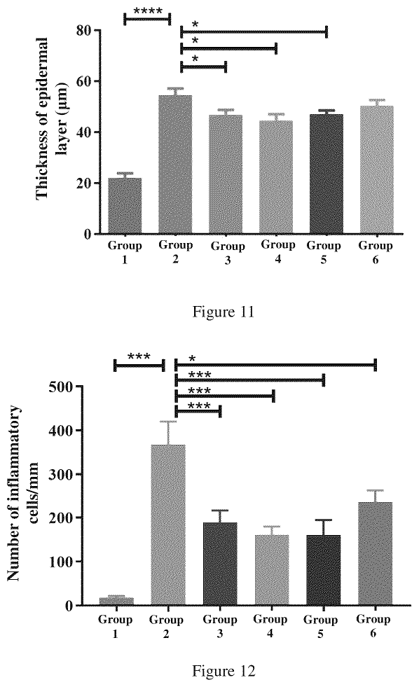

H&E staining of mouse right ear tissue sections is shown in FIG. 10. The thickness of the epidermal layer of the right ear of mice in each group was measured. The epidermal thickness of the right ear of the model group 2 was significantly increased relative to that in group 1 using One-way ANOVA analysis (****P<0.0001; FIG. 11; Table 12), and inflammatory cell infiltration such as eosinophils and macrophages was significantly increased (***P<0.001; FIG. 10; FIG. 12; Table 13).

Compared with the model group 2 administered with vehicle, compound 1 could significantly inhibit epidermal layer thickening of the right ear (p<0.05; FIG. 11; Table 12) and inflammatory cell infiltration (***p<0.001; FIG. 10; FIG. 12; Table 13) when it was administered at a dose of 3 mpk by intragastric administration once daily for 22 consecutive days; compound 1 significantly reduced the thickness of the epidermal layer of the right ear (*p<0.05; FIG. 11; Table 12) and significantly reduced inflammatory cell accumulation (***p<0.001; FIG. 10; FIG. 12; Table 13) when it was administered at a dose of 10 mpk by intragastric administration once daily for 22 consecutive days; compound 1 significantly reduced the epidermal thickness of the right ear (*p<0.05; FIG. 11; Table 12) and the number of inflammatory cells (***p<0.001; FIG. 10; FIG. 12; Table 13) when it was administered at a dose of 30 mpk by intragastric administration once daily for 22 consecutive days.

Compared with the model group 2 administered with vehicle, the positive control drug Abrocitinib significantly reduced inflammatory cell infiltration (*p<0.001; FIG. 10; FIG. 12; Table 13) but did not inhibit epidermal layer thickening of the right ear (p>0.05; FIG. 11; Table 12) when it was administered at a dose of 17 mpk by intragastric administration once daily for 22 consecutive days.

TABLE 12
Analysis of thickness of epidermal layer of
right ear of mice in each experimental group

| | | Significance |
|---|---|---|
| Group | p value | level |
| Group 1 control group | <0.0001 | **** |
| Group 2 modeling + vehicle, p.o. | — | — |
| Group 3 modeling + compound 1, 3 mpk, p.o. | 0.0333 | * |
| Group 4 modeling + compound 1, 10 mpk, p.o. | 0.0145 | * |
| Group 5 modeling + compound 1, 30 mpk, p.o. | 0.0281 | * |
| Group 6 modeling + Abrocitinib, 17 mpk, p.o. | 0.2627 | ns |

Note:
The thickness of the epidermal layer of the right ear in each experimental group was compared using One-way ANOVA analysis, and the p-values were all compared to that of group 2.

TABLE 13
Analysis of inflammatory cell content in right
ear tissue of mice in each experimental group

| | Number of inflammatory | |
|---|---|---|
| Group | cells per mm | Significance level |
| Group 1 control group | 18 ± 3.87 | *** |
| Group 2 modeling + vehicle, p.o. | 368 ± 51.76 | — |
| Group 3 modeling + compound 1, 3 mpk, p.o. | 189 ± 27.25 | *** |
| Group 4 modeling + compound 1, 10 mpk, p.o. | 162 ± 18.31 | *** |
| Group 5 modeling + compound 1, 30 mpk, p.o. | 160 ± 34.18 | *** |
| Group 6 modeling + Abrocitinib, 17 mpk, p.o. | 236 ± 26.59 | * |

Note:
Data are expressed as Mean ± SEM, and the significance levels were all compared to that of G2.

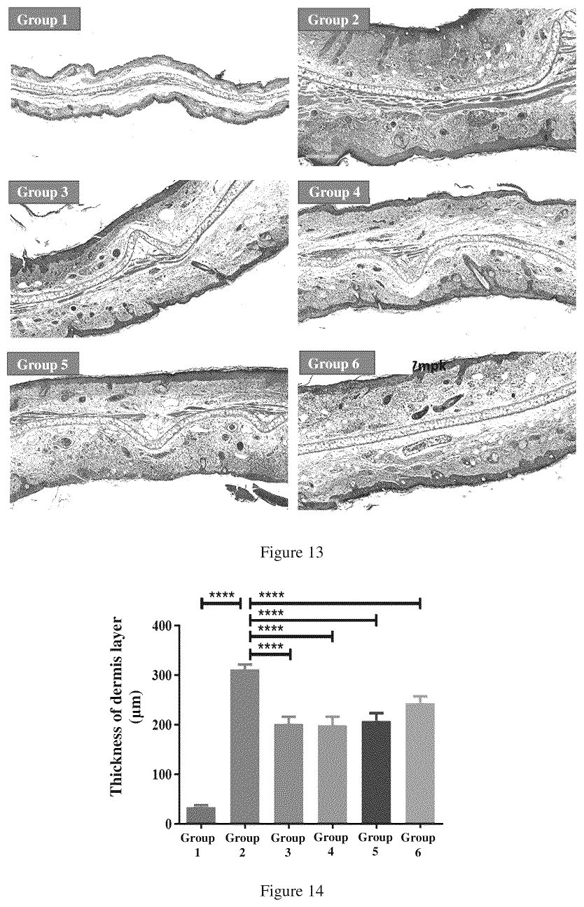

Masson staining of mouse right ear tissue sections is shown in FIG. 13. The thickness of the dermis layer of the right ear of mice in each group was measured. The dermis layer thickness of the right ear of the model group G2 was significantly increased relative to that in group 1 using One-way ANOVA analysis (****P<0.0001; FIG. 14; Table 14).

Compared with the model group 2 administered with vehicle, compound 1 could significantly inhibit dermis layer thickening of the right ear (****P<0.0001; FIG. 14; Table 14) when it was administered at a dose of 3 mpk by intragastric administration once daily for 22 consecutive days; compound 1 significantly reduced the thickness of the dermis layer of the right ear (****P<0.0001; FIG. 14; Table 14) when it was administered at a dose of 10 mpk by intragastric administration once daily for 22 consecutive days; compound 1 could significantly reduce the thickness of the dermis layer of the right ear (****P<0.0001; FIG. 14; Table 14) when it was administered at a dose of 30 mpk by intragastric administration once daily for 22 consecutive days.

Compared with the model group 2 administered with vehicle, the positive control drug Abrocitinib could significantly inhibit the increase of the thickness of the dermis layer of the right ear (***p<0.001; FIG. 14; Table 14) when it was administered at a dose of 17 mpk by intragastric administration once daily for 22 consecutive days.

TABLE 14
Analysis of thickness of dermis layer of right
ear of mice in each experimental group

| | | Significance |
|---|---|---|
| Group | p value | level |
| Group 1 control group | <0.0001 | **** |
| Group 2 modeling + vehicle, p.o. | — | — |
| Group 3 modeling + compound 1, 3 mpk, p.o. | <0.0001 | **** |
| Group 4 modeling + compound 1, 10 mpk, p.o. | <0.0001 | **** |
| Group 5 modeling + compound 1, 30 mpk, p.o. | <0.0001 | **** |
| Group 6 modeling + Abrocitinib, 17 mpk, p.o. | 0.0007 | *** |

Note:
The thickness of the dermis layer of the right ear in each experimental group was compared using One-way ANOVA analysis, and the p-values were all compared to that of group 2.

### CONCLUSION

Under the experimental conditions, after being orally taken once daily for 22 consecutive days, compound 1 at doses of 3 mpk, 10 mpk, and 30 mpk could significantly inhibit right ear pinna thickening, epidermal layer thickening, and dermis layer thickening in mice of the OXA-induced atopic dermatitis model, and could significantly reduce inflammatory cell infiltration. The clinical symptom scores of the OXA-induced mouse atopic dermatitis could be significantly inhibited when the doses were 10 mpk and 30 mpk. The compound had significant anti-inflammatory and atopic dermatitis symptom inhibiting effects.

The aforementioned examples and descriptions of certain embodiments should be construed as illustrating, rather than as limiting, the present invention as defined by the claims. As will be readily understood, many changes and combinations of the features described above can be employed without departing from the present invention as set forth in the claims. All such changes are intended to be included within the scope of the present invention. All cited references are incorporated herein by reference in their entirety.

1. (canceled)

2. A method for treating or preventing atopic dermatitis, comprising administering to a subject a therapeutically effective amount of compound 1 or a pharmaceutically acceptable salt of the compound 1, wherein the compound 1 has following structure:

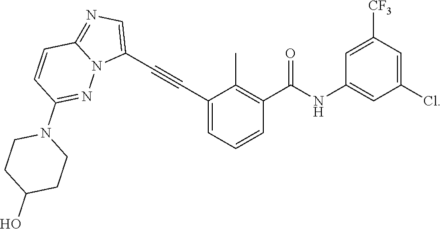 [SMILES: Cc1c(C#Cc2cnc3ccc(N4CCC(O)CC4)nn23)cccc1C(=O)Nc1cc(Cl)cc(C(F)(F)F)c1]

3. The method for treating or preventing atopic dermatitis according to claim 2, wherein the compound 1 or the pharmaceutically acceptable salt of the compound 1 is administered topically.

4. The method for treating or preventing atopic dermatitis according to claim 2, wherein the compound 1 or the pharmaceutically acceptable salt of the compound 1 is administered orally.

5. A pharmaceutical composition for treating or preventing atopic dermatitis, comprising compound 1 or a pharmaceutically acceptable salt of the compound 1, and a pharmaceutically acceptable excipient, wherein the compound 1 has following structure:

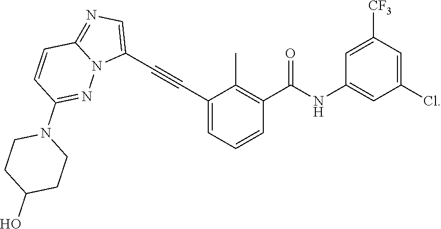 [SMILES: Cc1c(C#Cc2cnc3ccc(N4CCC(O)CC4)nn23)cccc1C(=O)Nc1cc(Cl)cc(C(F)(F)F)c1]

6. The pharmaceutical composition for treating or preventing atopic dermatitis according to claim 5, wherein the pharmaceutical composition is administered topically.

7. The pharmaceutical composition for treating or preventing atopic dermatitis according to claim 5, wherein the pharmaceutical composition is administered orally.

8. Compound 1 or a pharmaceutically acceptable salt of the compound 1 for use in treatment or prevention of atopic dermatitis, wherein the compound 1 has following structure:

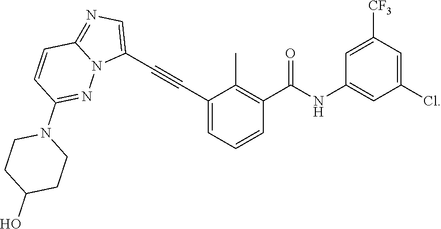 [SMILES: Cc1c(C#Cc2cnc3ccc(N4CCC(O)CC4)nn23)cccc1C(=O)Nc1cc(Cl)cc(C(F)(F)F)c1]

9. The compound 1 or the pharmaceutically acceptable salt of the compound 1 according to claim 8, wherein the compound 1 or the pharmaceutically acceptable salt of the compound 1 is administered topically.

10. The compound 1 or the pharmaceutically acceptable salt of the compound 1 according to claim 8, wherein the compound 1 or the pharmaceutically acceptable salt of the compound 1 is administered orally.
